(19) United States
(12) Patent Application Publication
(10) Pub. No.: US 2002/0155182 A1
(43) Pub. Date: Oct. 24, 2002

(54) Chia seed beverage and method

(76) Inventor: David Belna, Costa Mesa, CA (US)

Correspondence Address: Ray K. Shahani, Esq., Attorney at Law, Twin Oaks Office Plaza, 477 Ninth Avenue, Suite 112, San Meteo, CA, 94402-1854, US

(21) Appl. No.: 10/093,956

(22) Filed: Mar. 7, 2002

Related U.S. Application Data
(60) Provisional application No. 60/274,307, filed on Mar. 8, 2001.

(51) Int. Cl. A61K 35/78

(52) U.S. Cl. 424/746; 424/750

(57) ABSTRACT

A beverage including an extract from Chia seed, water, sweetener and a preservative. The sweetener is selected from the group consisting of: maple syrup; corn syrup; fruit juice; honey; sucrose syrup; and glucose-fructose syrup. The Chia seed extract forms a gel in the digestive tract to slow the absorption of carbohydrates, namely sugars into the blood stream to maintain normal blood sugar levels in the beverage consumer.

### RELATED INVENTIONS

This Application is related to and incorporates herein by reference, in its entirety, U.S. Provisional Application Serial No. 60/274,307 filed Mar. 8, 2001 entitled CHIA SEED BEVERAGE AND METHOD, and claims any and all benefits to which it is entitled therefrom.

### FIELD OF THE INVENTION

The present invention relates beverages that optimize the digestion of carbohydrates in humans, more particularly this invention relates to beverages including Chia seed, Chia gel, or other Chia derivatives.

### BACKGROUND OF THE INVENTION

There are various types of beverages. Some beverages are developed to hydrate a person during and after exercise, vigorous work, or exertion. Such drinks typically include a water base with a sweetener, a flavoring, and some electrolyte. Accordingly, fluid lost due to perspiration and respiration is replaced and electrolyte balance can be restored. One drawback of a popular sports drink, Gatorade®, is that nearly all of the calories supplied are derived from carbohydrates, and virtually all of these carbohydrates are sugars.

Other beverages combining various juices, concentrates, and vitamins in order to enhance one's health. Odwalla®, for example, is a brand name for a line of beverages that use fruit juice as a base, and add various vitamins, herbs and supplements to make the drinks nutritious. One drawback to these and other similar beverages is that at least 75% of the calories are from carbohydrates, primarily sugars. The “C Monster” beverage, distributed by Odwalla®, includes on a per serving basis, 5 grams of fat, 2 grams of protein, and 34 grams of carbohydrate. Of these 34 grams of carbohydrates, 27 grams are sugar.

The “Super Protein”™ protein formulation beverage offered by Odwalla®, includes 6 grams of protein per serving, and only 32 grams of carbohydrate. This shift in the ratio of protein to carbohydrate content is accomplished by the addition of soy protein.

Various other bottled beverages having fruit juice or concentrated juice as a main ingredient have one thing in common: nearly all of the calories are derived from carbohydrates, namely sugar. Sugar is known to be a direct cause of fluctuations in blood glucose (sugar) levels.

Fluctuations in blood glucose levels are undesirable for several reasons. Many experience a sugar-high after ingesting refined carbohydrates or sugars only to feel tired a short period thereafter. When the body detects relatively high blood glucose levels, insulin is treated to reduce a level blood sugar. Sometimes either too much or too little insulin is treated causing non-optimal blood sugar levels.

Another reason that fluctuations in blood glucose levels are undesirable is that athletes particularly those that engage in biking, running, and other endurance sports, require constant blood glucose levels in order to achieve optimal performance and optimal endurance.

Research reveals that over half of all deaths in United States are diet-related, that is, the cause of death has been identified as having statistical correlation to a particular dietary scheme. Metabolic diseases including cardiovascular disease and diabetes mellitus had been statistically linked to dietary habits, including excessive consumption of refined carbohydrates and sugars. Cardiovascular disease is the top killer. Cancer is second. Diabetes and its complications are the third leading cause of death and Canada and United States.

According to a report issued by the National Commission on Diabetes, as many as 10 million North Americans may have diabetes, and new incidents increase yearly. Although the acute and often lethal symptoms of diabetes can be controlled by insulin therapy, the long-term complications may reduce life expectancy, as much is ⅓. Compared with rates of incidents in non-diabetic persons, diabetic patients show rates which are increased for five-fold for blindness, seventeen-fold for kidney disease, five fold for gangrene, and two-fold for heart disease.

There are two major forms of diabetes, Type I diabetes, which is also known as insulin dependent diabetes mellitus, and Type II diabetes. Most patients with Type I diabetes have a common pathological picture: the nearly total disappearance of insulin producing pancreatic data cells which results in hyperglycemia.

Type II diabetes, also known as not insulin dependent diabetes, is more common than type I diabetes, affecting 90 percent of all persons with diabetes. Type II diabetes is a mild form of diabetes, and often of gradual onset, which usually appears in middle age. Fortunately, abnormal plasma glucose levels seen in Type II diabetes can usually be controlled by diet alone.

A low carbohydrate diet, for example, has been used to regulate Type II diabetes. Unfortunately, not all patients are willing to radically change their diets and significantly cut their carbohydrate intake. Also, there are long-term health risks associated with high protein diet including health risks linked with excessive intake of meat products, which, may to contribute to the onset of coronary, and other types of disease.

U.S. Pat. No. 4,801,582 discloses a method for treating hypoglycemia using aloe polysaccharides. This U.S. patent is incorporated herein by reference. While the aloe polysaccharides appear somewhat effective at moderating plasma glucose levels, there is still in need for improved methods of treating hypoglycemia and other metabolic disease.

Effective ways to moderate the digestion of starches and sugars is desired. What is also desired is a beverage which is rich in protein and useful fatty acids and which minimizes fluctuation in blood glucose levels during and after consumption. What is also desired is a beverage which helps athletes, particularly endurance athletes, achieve optimal performance.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

Chia seed is a source of dietary protein providing all the essential amino acids. Compared to other seeds and grains, Chia seed is a significant source of protein, being between 19 to 23 percent of protein by weight. Chia seed is also a significant source of fiber.

One exceptional quality of Chia seed is its ability to absorb nearly twelve times its weight in water. This ability to hold water can prolong hydration and retain electrolyte's can body fluids, especially during exertion. Normal fluid retention insures normal electrolyte dispersion across all membranes, maintains fluid balances and aids normal cellular function.

Chia seeds contain water soluble fiber that, when wet, produces a gel. This gel protects the Chia seed against drying in arid climates where the Chia plants naturally grow.

According to one method of this invention, a spoon full of Chia seed is mixed in a glass of water and left for thirty minutes to form a gel. Researchers believe that the Chia gel also forms in the digestive tract when eaten. The gel is thought to create a physical barrier between various starches, sugars and other carbohydrates consumed by a person and the various digestive enzymes that break these carbohydrates down. The Chia seeds slow the conversion of carbohydrates into sugar, normalizes blood glucose fluctuations and thereby improves athletic endurance.

Chia seed contains three to ten times the oil concentrations of many grains. The unsaturated fatty acids in Chia seed are essential oils the body needs to help emulsified an absorb the fact soluble vitamins A, and D, E and K. Chia seeds and gel are rich in the essential unsaturated fatty acids. Chia seed is a rich source of calcium and also contains boron, which helps body to absorb and utilize the available calcium.

Various compounds, mixtures and materials derived from the naturally occurring Chia seed, or synthetic derivatives thereof. These substances regulate the digestion of carbohydrates to achieve improved nutrition and to maintain healthy blood glucose levels.

The Chia seed is a seed from the family labiatae, commonly referred to as Mint. There are two particular families of Chia seed, columbaraie, and salvia hispanica. The columbaraie plants are indigenous to the Southwestern United States, and grow in the desert climate. The plant salvia hispanica is native to southern Mexico and Guatemala.

The Chia seed gel is formed by adding water to seeds, stirring, and allowing soluble fiber from the seeds to gel. Normally, 7-12 parts water to one part Chia seed (by volume) provides sufficient hydration to form Chia seed gel.

In theory, there are numerous possible explanations for how the Chia seed Gel and various derivatives work. One possible explanation is that the Chia seed gel coats the linings of the digestive track and physically inhibits the assimilation of carbohydrates, or shields the carbohydrates from digestive enzyme action.

Another explanation, is that the Chia seed gel absorbs significance amounts of water, inhibiting hydrolysis, which inhibits the digestion of carbohydrates. A further explanation of is that the Chia seed gel contains particular polysaccharides that bond with various monosaccharides and disaccharides within the digestive system, thereby inhibiting or slowing the digestion of complex carbohydrates and refined sugars.

There are other explanations, including amylase inhibition properties of the Chia seed gel, and by bio-adhesive of properties of the Chia seed gel. It is also possible that the Chia seed gel inhibits detection of monosaccharides and disaccharides by receptors in the walls of the digestive track and thereby inhibits secretion of amylase and other digestive enzymes.

A method of preparing the Chia seeds for use in a beverage includes providing Chia seeds having a seed body, adding water to the Chia seeds to form a gel, separating the seed body from the gel, and mixing the gel with fruit juice concentrate. The gel/fruit juice mixture is pasteurized. The pasteurized mixture is frozen to make fruit pops; or packaged to make non-frozen fruit/gel packs. According to one aspect of the invention, the gel/fruit juice mixture is dehydrated to make flat fruit leather and/or licorice stick facsimile candy.

Chia seeds are processed in any of a number of ways. According to one aspect of the invention water is added to the seeds so that the gel forms and then the gel is cold press to separate the seed body from the gel. Rotating a centrifuge is an effective alternative to separate the seed body from the gel.

Adding a solvent to the Chia seed gel effectuates processing of the Chia seeds. The Chia seed gel can be reduced and encapsulating for oral delivery such as desirable when using the gel as a dietary supplement or in accordance with pharmaceutical purposes.

It can be appreciated that an alternate method in accordance with the present invention includes a bio-adhesive to the Chia seed gel, or derivative thereof. U.S. Pat. No. 6,123,965, the disclosure of which is incorporated herein by reference, discloses various methods and compositions for enhancing the bio-adhesive properties of bio -compatible materials. Preferably, the bio-adhesive includes a metal compound such as a metal compound selected from the group consisting of: calcium oxide; iron oxide; copper oxide; and zinc oxide.

According another aspect of the invention and amylase inhibitor is added to the Chia seed gel, an ingested. Including bio-adhesives in beverages is intended to prevent the onset of metabolic disease, to improve digestion and to improve nutrition.

An amylase inhibitor of wheat origin is disclosed in U.S. Pat. No. 5,332,803, the disclosure of which is incorporated herein by reference. Accordingly, this amylase inhibitor inhibits the hydrolysis of carbohydrate into glucose thereby moderating increases in blood glucose levels, ultimately minimizing resultant insulin secretions. It can be appreciated that any of a number of synthetic amylase inhibitors, and natural ones, can be substituted for the wheat originating amylase inhibitor in accordance with the present invention.

### Additional Compositions

A composition, in accordance with the present invention includes the steps comprising:

(a) providing Chia seeds, having a seed body;

(b) adding water to the Chia seeds to form a gel;

(c) separating the seed body from the gel; and

(d) collecting the gel.

The seed body is separated from the gel by pressing the gel, or by centrifugation. An amylase inhibitor, or a bio-adhesive, or both can be added to the composition.

The separated gel can be reduced and added to a carbohydrate-based food such as breads; beverages; crackers; potato chips; synthesized snack foods; and drinks.

Under some circumstances separation of useful polysaccharides from the composition is desirable. Accordingly, the steps of extracting at least one polysaccharide from the gel and collecting the polysaccharide are performed.

A pharmaceutical composition useful in the treatment of diabetes, hyperglycemia, hypoglycemia or obesity is derived in accordance with the present invention. A pharmaceutically compatible excipient is added to form the pharmaceutical composition. Further, a pharmaceutically compatible carrier is added. One aspect of the invention of the pharmaceutical composition includes an amylase inhibitor, or a bio-adhesive, or both, added to the composition.

Under various circumstances it is desirable to include the seed body with the composition for nutritional value and for increased fiber content of the composition. Under other circumstances it is desirable to retain all components of the Chia gel including pectin, oils, minerals, complex carbohydrates, and other components. Reducing the Chia gel into more fundamental compositions is often desirable.

One exemplary composition is prepared by the steps comprising:

(a) providing Chia seeds, having a seed body;

(b) rinsing the Chia seeds with water to form a gel; and

(c) separating the seed body from the gel by collecting the water.

In nature, the gel normally adheres to the Chia seed body after water is added to the seed, and the gel is challenging to thereafter remove. The present method includes quickly rinsing the seeds to wisk incremental portions of gel-forming water soluble fiber from the seeds, before the gel forms and adheres to the seed body. This is best accomplished in cold water because warm water accelerates gel formation.

The method step of separating includes rinsing the seeds with water at having temperature below 65 degrees, and preferably water having a temperature between 40-60 degrees Fahrenheit to inhibit gel formation on the seed body.

The rinsing will occur during a period of less than 90 seconds to inhibit gel formation on the seed body. Optimally the period is between 30-60 seconds to inhibit gel formation on the seed body. Of course, the time for rinsing can be modified in accordance with the water temperature.

According to one aspect of the invention where hot water (greater than room temperature) is used, the rinsing period is very short e.g. less than 30 seconds.

In any event, the rinsing process repeats to optimize yield. According to one aspect of the invention, the total volume of rinse water is approximately ten times the volume of seed. This enables the rinse water to be collected and enables the gel to form in the rinse water without an excess of, or a shortage of, water. The rinsing process optimally repeats between 6-10 times, but this number can be modified according to the temperature of the water and the rinse period length.

A solvent can be added to the seed, or to the rinse water, to prevent the gel from forming on the seed. Solvents may include alcohol, or other known solvent.

The seeds are agitated during the rinsing process. According to one aspect of the invention, the seeds are slowly centrifuged. Alternatively, a shaker shakes the seeds during rinsing.

The composition, if desired to be reduced from a gel, or if a gel has not optimally formed, is dried at low temperature in a vacuum to minimize any loss of efficacy and nutrition. Preferably, the gel or solution including the water soluble fiber is collected in the vacuum and dried at a temperature of less than 200 degrees Fahrenheit.

A method in accordance with the invention includes crushing the seeds before the step of rinsing to optimize the surface area of the seed and to improve the soluble fiber gathering process.

An example of a method that works include preparing a composition prepared by the steps comprising:

(a) providing Chia seeds, having a seed body;

(b) rinsing the Chia seeds with in water having a volume of no more than two times the Chia seed volume;

(c) collecting the water; and

(d) repeating the step (b) and the step (c), whereby gel forms in the collected water.

Though the present invention is described in terms of treating human patients, it should be known that the invention can be practiced to treat animals, particularly mammals. The Chia seed and its derivatives can be used as a food supplement to enhance the diet and regulate the digestion in livestock. Further, the composition and method of the present invention are useful for cosmetic products, and a fat substitute in foods.

Unless defined otherwise, all technical and scientific terms used herein have the same meaning as commonly understood by one of ordinary skill in the art to which the present invention belongs. Although any methods and materials similar or equivalent to those described can be used in the practice or testing of the present invention, the preferred methods and materials are now described. All publications and patent documents referenced in the present invention are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

We claim:

1. A beverage, comprising:
water;
sweetener;
preservative;
an extract from Chia seed.

2. A beverage as set forth in claim 1, wherein the sweetener is selected from the group consisting of: maple syrup; corn syrup; fruit juice; honey; sucrose syrup; and glucose-fructose syrup.

3. A beverage as set forth in claim 1, wherein the preservative is selected from the group consisting of: ascorbic acid and citric acid.

4. A beverage as set forth in claim 1, wherein the extract is Chia seed gel.

5. A beverage as set forth in claim 1, wherein Chia seed is from the family Salvia Hispanica.

6. A beverage as set forth in claim 1, further comprising an amylase inhibitor.

7. A beverage as set forth in claim 1, further comprising an amylase inhibitor of wheat origin.

8. A beverage as set forth in claim 1, further comprising a bio adhesive.

9. A beverage as set forth in claim 1, further comprising a bio adhesive selected from the group consisting of: calcium oxide; iron oxide; copper oxide; and zinc oxide.

10. A beverage as set forth in claim 1, further comprising fruit juice concentrate.

11. A beverage as set forth in claim 1, wherein one serving of the beverage is 8 fluid ounces having at least 25 grams of sugar.
